(19) United States
(12) Patent Application Publication
(10) Pub. No.: US 2018/0104093 A1
(43) Pub. Date: Apr. 19, 2018

(54) ELBOW HEATING SLEEVE

(71) Applicant: Jamie Jencks, Warwick, RI (US)

(72) Inventor: Jamie Jencks, Warwick, RI (US)

(21) Appl. No.: 15/296,531

(22) Filed: Oct. 18, 2016

(51) Int. Cl. A61F 7/00; A61F 7/02

(52) U.S. Cl. CPC A61F 7/007; A61F 2007/0078; A61F 2007/0032; A61F 7/02

(57) ABSTRACT

A therapeutic sleeve that includes: an opening adapted to receive a user's elbow; a plurality of heating elements integrated into the sleeve; and a power source, where the power source provides energy for the plurality of heating elements. The power source includes a battery or a AC power plug.

### BACKGROUND OF THE INVENTION

### Field of Invention

The present invention relates to an elbow heating sleeve used for therapeutic purposes.

### Description of Related Art

Many times individuals and athletes suffer elbow pain or injury. This elbow pain may be treated by heating or ice treatment to alleviate the pain and swelling that's usually associated with an elbow injury. Ice treatment is normally used to reduce swelling and heat treatments increase blood flow to the area to help heal and alleviate the injury. Several ways have been developed to introduce both heating and cooling to the elbow area. Further various wraps and sleeves have been developed to place over the elbow area to also assist in reducing swelling. However many times further devices are needed to address elbow injuries and therapeutic application of heat to the area.

### SUMMARY OF THE INVENTION

The present invention relates to a therapeutic sleeve that includes: an opening adapted to receive a user's elbow; a plurality of heating elements integrated into the sleeve; and a power source, where the power source provides energy for the plurality of heating elements. The power source includes a battery or a AC power plug.

### BRIEF DESCRIPTION OF DRAWINGS

FIG. 1 depicts use of an elbow heating sleeve in accordance with the present invention.

### DETAILED DESCRIPTION

The present invention relates to an elbow heating sleeve that includes electronic mechanisms to provide radiant heat to the elbow area. The elbow heating sleeve includes an integrated heating element and may be charged electrically with a battery enclosed therein or through the use of a AC cord attached thereto.

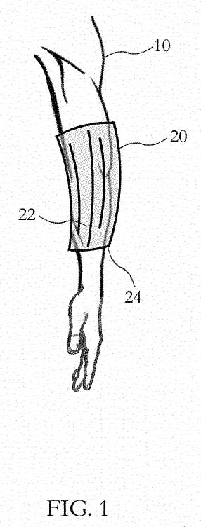

In reference to FIG. 1 an application of an elbow heating sleeve 20 in accordance with the present invention is depicted. The heating sleeve 20 as shown in FIG. 1 slides over an individual's arm covering the elbow and surrounding regions. The sleeve 20 includes integrated heating elements 22 within the material of the heating sleeve 20. An opening 24 is provided for the insertion of a user's hand and arm to place the elbow heating sleeve 20 into place. Preferably, the heating sleeve 22 includes battery power or alternatively may be electrically connected to a AC outlet through removable and temporary wiring. An opening 24 is provided through the sleeve 20 for the user to insert the sleeve into place over the user's elbow. Preferably the heating sleeve 20 also includes a waterproof covering and is made of lightweight materials to apply heat to the elbow region. This application of heat as expected encourages the blood flow and release of tension and pain in the elbow area. The instant invention has been shown and described in what it considers to be the most practical and preferred embodiments. It is recognized, however, that departures may be made there from within the scope of the invention and that obvious modifications will occur to a person skilled in the art.

What is claimed is:

1. A therapeutic sleeve comprising:
an opening adapted to receive a user's elbow;
a plurality of heating elements integrated into the sleeve; and
a power source, where the power source provides energy for the plurality of heating elements.

2. The therapeutic sleeve according to claim 1, where the power source includes a battery.

3. The therapeutic sleeve according to claim 1, where the power source includes a AC power plug.
